(19) United States
(12) Patent Application Publication
(10) Pub. No.: US 2024/0295630 A1
(43) Pub. Date: Sep. 5, 2024

(54) SENSOR SYSTEM, VEHICLE COMPRISING SAID SENSOR SYSTEM, AND RADIO WAVE TRANSMITTING AND RECEIVING METHOD

(71) Applicant: Murata Manufacturing Co., Ltd., Nagaokakyo-shi (JP)

(72) Inventor: Daichi UEKI, Nagaokakyo-shi (JP)

(73) Assignee: Murata Manufacturing Co., Ltd., Nagaokakyo-shi (JP)

(21) Appl. No.: 18/661,709

(22) Filed: May 13, 2024

Related U.S. Application Data
(63) Continuation of application No. PCT/JP2022/043601, filed on Nov. 25, 2022.

(30) Foreign Application Priority Data
Dec. 10, 2021 (JP) 2021-200762

(51) Int. Cl. G01S 7/40; A61B 5/18; G01S 13/86; G01S 13/88

(52) U.S. Cl. CPC G01S 7/4013; G01S 13/862; G01S 13/88; A61B 5/18

(57) ABSTRACT

A sensor system that controls a sampling frequency of electromagnetic waves that a radio wave sensor emits and reduces electric power consumption, a vehicle that includes this sensor system, and a radio wave transmitting and receiving method of the sensor system are provided. The sensor system is configured to include a radio wave sensor, a vibration sensor, and a signal processing device. The radio wave sensor emits electromagnetic waves toward a measuring object, receives reflected waves, and outputs reflected wave data to the signal processing device. The vibration sensor measures vibration to be superposed on the reflected waves received by the radio wave sensor as noise and outputs measured vibration data to the signal processing device. The signal processing device recognizes a frequency range of the vibration measured by the vibration sensor, identifies an upper limit of the frequency range, and sets the sampling frequency of radio waves.

### CROSS-REFERENCE TO RELATED APPLICATIONS

This is a continuation application of PCT/JP2022/043601, filed on Nov. 25, 2022, designating the United States of America, which is based on and claims priority to Japanese Patent Application No. JP 2021-200762, filed on Dec. 10, 2021. The entire contents of the above-identified applications, including the specifications, drawings and claims, are incorporated herein by reference in their entirety.

### TECHNICAL FIELD

The present disclosure relates to a sensor system that emits electromagnetic waves from a radio wave sensor and receives reflected waves returned from a measuring object, a vehicle including this sensor system, and a radio wave transmitting and receiving method.

### BACKGROUND ART

Prior art sensor systems of this kind include, for example, a sensor system disclosed in patent document 1.

This sensor system includes a radio wave sensor, a vibration sensor, and a signal processing device. The radio wave sensor transmits radio waves to a detection area, receives radio waves reflected off an object, and outputs a radio wave sensor signal that corresponds to the state of the object to the signal processing device. The vibration sensor detects vibration of at least one of the radio wave sensor and the object and outputs a vibration sensor signal that corresponds to detected vibration to the signal processing device. The signal processing device attenuates a vibration component detected using the vibration sensor signal from the radio wave sensor signal and generates a signal that mostly includes the component of the object.

### CITATION LIST

### Patent Document

Patent Document 1: Japanese Unexamined Patent Application Publication No. 2021-71326

### SUMMARY OF DISCLOSURE

### Technical Problem

However, the foregoing prior art sensor system disclosed in the patent document 1 empirically determines the upper limit of the frequency of the vibration sensor signal detected by the vibration sensor. Then, the radio wave sensor is operated by setting a sampling frequency of radio waves emitted from the radio wave sensor to a sampling frequency that enables the vibration sensor signal having the empirically determined frequency to be sufficiently acquired. Because of this, in the foregoing prior art sensor system, depending on the vibration component superposed on the radio wave sensor signal, the sampling frequency of radio waves emitted from the radio wave sensor is set to an excessively high value, and electric power is consumed wastefully.

### Solution to Problem

The present disclosure is made to resolve such issues and configures a sensor system including:
a radio wave sensor that emits an electromagnetic wave toward a measuring object and receives a reflected wave reflected assuming the electromagnetic wave hits the measuring object;
a vibration sensor that measures vibration to be superposed on the reflected wave as noise; and a sampling frequency setting part that recognizes a frequency range of the vibration, identifies an upper limit of the frequency of the vibration, determines that a sampling frequency of the electromagnetic wave that the radio wave sensor emits is a sampling frequency whose Nyquist frequency is equal to an identified upper limit of the frequency, and sets the sampling frequency of the electromagnetic wave that the radio wave sensor emits to a determined sampling frequency.

Further, the present disclosure configures a radio wave transmitting and receiving method including:
a vibration measuring step that emits an electromagnetic wave toward a measuring object from a radio wave sensor and measures vibration to be superposed on the reflected wave reflected assuming the electromagnetic wave hits the measuring object as noise;
a vibration frequency upper limit identifying step that recognizes a frequency range of the vibration and identifies an upper limit of the frequency of the vibration; a sampling frequency determining step that determines that a sampling frequency of the electromagnetic wave that the radio wave sensor emits is a sampling frequency whose Nyquist frequency is equal to an identified upper limit of the frequency; and
a sampling frequency setting step that sets the sampling frequency of the electromagnetic wave to the sampling frequency determined by the sampling frequency determining step.

According to these configurations, the sampling frequency setting part or the vibration frequency upper limit identifying step recognizes the frequency range of the vibration to be superposed on the reflected wave of the electromagnetic wave as noise, which is received by the radio wave sensor, and identifies the upper limit of the frequency of the vibration to be superposed as noise. Subsequently, the sampling frequency setting part or the sampling frequency determining step determines that the sampling frequency of the electromagnetic wave that the radio wave sensor emits is the sampling frequency whose Nyquist frequency is equal to the identified upper limit of the frequency. The sampling frequency setting part or the sampling frequency setting step sets the sampling frequency of the electromagnetic wave that the radio wave sensor emits to the determined sampling frequency.

Accordingly, this enables the radio wave sensor to emit the electromagnetic wave with the sampling frequency determined based on the vibration that is actually detected by the vibration sensor. Because of this, electric power is not consumed wastefully like the prior art technology in which the electromagnetic wave with an excessively high sampling frequency is emitted from the radio wave sensor, and electric power consumption of the sensor system can be reduced.

Further, the present disclosure configures a vehicle including the sensor system described above.

According to the present configuration, it becomes possible to provide the vehicle including the sensor system that can reduce electric power consumption.

### Advantageous Effects of Disclosure

According to the present disclosure, it becomes possible to provide a sensor system that controls the sampling frequency of electromagnetic waves that a radio wave sensor emits and reduces electric power consumption, a vehicle that includes this sensor system, and a radio wave transmitting and receiving method.

### BRIEF DESCRIPTION OF DRAWINGS

FIG. 1 is a block diagram illustrating a schematic configuration of a sensor system according to a first embodiment of the present disclosure.

FIG. 2 is a flowchart illustrating a process of a radio wave transmitting and receiving method according to one embodiment of the present disclosure.

FIG. 3 is a diagram illustrating an exemplary relationship between the frequency of vibration measured by the sensor system illustrated in FIG. 1 and the frequency of a vital sign.

FIG. 4 is a perspective view of an interior of a vehicle that includes the sensor system illustrated in FIG. 1 as a driver monitoring system.

FIG. 5 is a block diagram illustrating a schematic configuration of a sensor system according to a second embodiment of the present disclosure and a configuration of a vibration component removing part.

FIG. 6 is a block diagram illustrating a schematic configuration of a sensor system according to a third embodiment of the present disclosure.

FIG. 7 is a block diagram illustrating a schematic configuration of a sensor system according to a fourth embodiment of the present disclosure.

### DESCRIPTION OF EMBODIMENTS

Next, embodiments of a sensor system of the present disclosure, a vehicle including this sensor system, and a radio wave transmitting and receiving method are described.

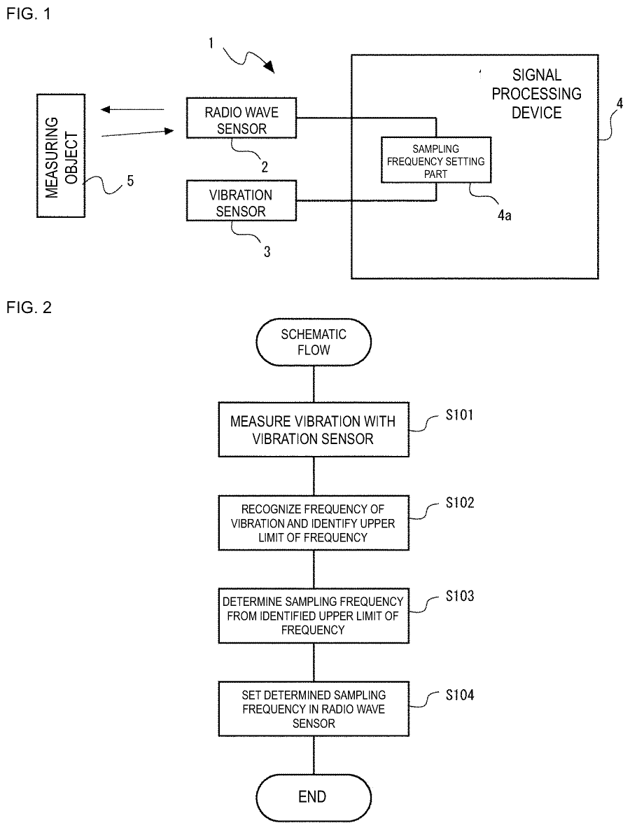

FIG. 1 is a block diagram illustrating a schematic configuration of a sensor system 1 according to a first embodiment of the present disclosure. The sensor system 1 is configured so as to include a radio wave sensor 2, a vibration sensor 3, and a signal processing device 4.

The radio wave sensor 2 emits electromagnetic waves toward a measuring object 5, receives reflected waves reflected assuming the electromagnetic waves hit the measuring object 5 (incoming reflection), and outputs received reflected wave data to the signal processing device 4. From these reflected waves, the radio wave sensor 2 detects a state of the measuring object 5, for example, a body surface displacement of the human body or the like. The radio wave sensor 2 is configured using, for example, a doppler radar, a FMCW (Frequency Modulated Continuous Wave) radar, a pulse radar, or the like. In the present embodiment, it is described that electromagnetic waves emitted from the radio wave sensor 2 are radio waves. However, the electromagnetic waves include various waves such as sound waves, light waves, and the like.

The vibration sensor 3 measures vibration to be superposed on the reflected waves as noise, which are received by the radio wave sensor 2, and outputs measured vibration data to the signal processing device 4. This vibration sensor 3 is configured using, for example, a six-axis inertial sensor, a three-axis acceleration sensor, or the like.

The signal processing device 4 includes a sampling frequency setting part 4a. The sampling frequency setting part 4a recognizes a frequency range of vibration measured by the vibration sensor 2 and identifies an upper limit of the frequency of the vibration, as described below. Subsequently, the sampling frequency setting part 4a determines that the sampling frequency of electromagnetic waves that the radio wave sensor 3 emits is the sampling frequency whose Nyquist frequency is equal to the identified upper limit of the frequency. Subsequently, the sampling frequency setting part 4a sets the sampling frequency of radio waves that the radio wave sensor 2 emits to the determined sampling frequency. Note that the sampling frequency is not necessarily calculated each time. In the case where a target for which the sensor system 1 is used is already determined, a value of the sampling frequency for this target may be measured and stored in advance.

FIG. 2 is a schematic flowchart of a radio wave transmitting and receiving method in the case where this radio wave transmitting and receiving method according to one embodiment of the present disclosure is applied to the foregoing sensor system 1. Steps S101 to S104 to be performed in this radio wave transmitting and receiving method are implemented by a CPU (central processing unit) included in the signal processing device 4 based on a computer program stored in a memory included in the signal processing device 4.

In the radio wave transmitting and receiving method of the sensor system 1, first, a vibration measuring step S101 that measures vibration using the vibration sensor 3 is performed. That is to say, the vibration sensor 3 measures vibration to be superposed on reflected waves as noise. These reflected waves are reflected assuming electromagnetic waves emitted from the radio wave sensor 2 hit the measuring object 5 and received by the radio wave sensor 2. The vibration sensor 3 outputs measured vibration data to the sampling frequency setting part 4a.

Next, a vibration frequency upper limit identifying step S102 that recognizes the frequency of the vibration and identifies the upper limit of the frequency is performed. That is to say, the sampling frequency setting part 4a recognizes a frequency range of the vibration measured by the vibration sensor 3, and the upper limit of the frequency of the vibration to be superposed on the reflected waves as noise, which are received by the radio wave sensor 2, is identified.

Next, a sampling frequency determining step S103 that determines a sampling frequency fₛ of the radio wave sensor 2 from the identified upper limit of the frequency is performed. That is to say, it is determined that the sampling frequency fₛ of radio waves that the radio wave sensor 2 emits is a sampling frequency fₛ whose Nyquist frequency fₙ is equal to an upper limit frequency fᵤₚₚₑᵣ identified by the vibration frequency upper limit identifying step S102.

Next, a sampling frequency setting step S104 that sets the radio wave sensor 2 in such a manner as to have the determined sampling frequency fₛ is performed. That is to say, the sampling frequency fₛ of radio waves that the radio wave sensor 2 emits is set to the sampling frequency fₛ determined by the sampling frequency determining step S103. This enables the radio wave sensor 2 to emit radio waves to the measuring object 5 with the sampling frequency fₛ.

Then, the radio wave sensor 2 emits radio waves toward the measuring object 5, and the radio wave sensor 2 receives reflected waves reflected assuming the radio waves hit the measuring object 5. The radio wave sensor 2 outputs received reflected wave data to the sampling frequency setting part 4a.

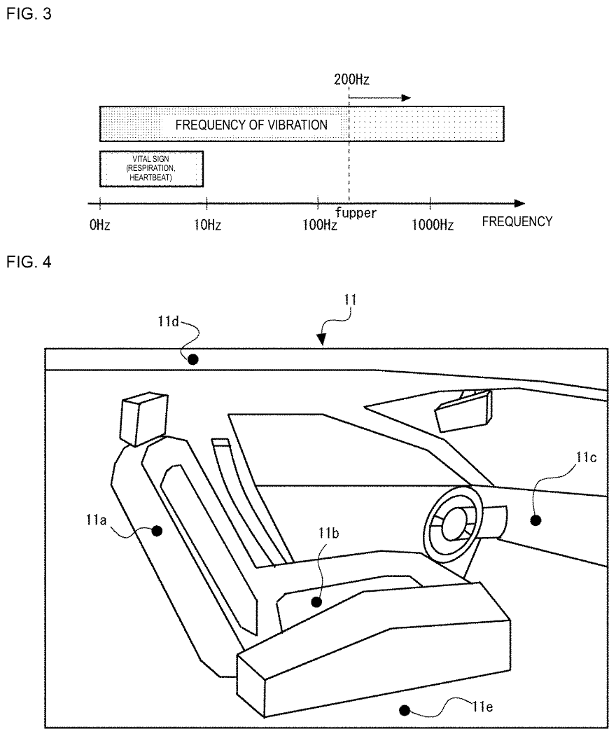

The identification of the upper limit of the frequency of the vibration in the vibration frequency upper limit identifying step S102 is performed, for example, by analyzing frequency information obtained by performing Fourier transformation on the vibration data and the intensity of the vibration data at each frequency. In the case where the measuring object 5 is a human body and a vital sign such as respiration or heartbeat of the human body is detected by measuring the body surface displacement of the human body, the frequency of a vital sign component measured by the radio wave sensor 2 becomes about 0 to 10 Hz as illustrated in FIG. 3. That is to say, the frequency of the measuring object 5, which is, for example, the frequency of the body surface displacement, becomes 0 to several hundred Hz or less than a frequency of vibration such as several thousand Hz in many cases.

In such cases, the reflected wave data are analyzed by extracting a frequency component of about 0 to 10 Hz, which corresponds to the measuring object 5, from the reflected wave data through a low pass filter. At that time, a vibration component is attenuated or removed from the reflected wave data that includes the vibration as noise to a level where body surface displacement data or the like can be sufficiently extracted. The signal processing is performed in the state where the vibration data is reduced as described above. Because of this, within the frequency range of the vibration recognized by the vibration sensor 3, of the frequency information obtained by performing Fourier transformation, from frequency components that exceed an intensity threshold value Pth, above which this signal processing can sufficiently extract the body surface displacement data or the like, the highest vibration frequency is searched. Subsequently, this highest vibration frequency is identified as the upper limit of the vibration frequency.

For example, with the relationship between the frequency of the vibration and the frequency of the vital sign illustrated in FIG. 3 as an example, if no vibration frequency with the intensity exceeding the threshold value Pth is included in a frequency of 200 Hz and above, the vibration frequency upper limit identifying step S102 identifies 200 Hz as the upper limit frequency fᵤₚₚₑᵣ.

Accordingly, in this case, the sampling frequency determining step S103 determines that the sampling frequency fₛ of radio waves that the radio wave sensor 2 emits is the sampling frequency fₛ whose Nyquist frequency fₙ is the identified upper limit frequency fᵤₚₚₑᵣ or 200 Hz, that is, 400 Hz (=200 Hz×2).

As described above, with the sensor system 1 according to the first embodiment illustrated in FIG. 1, which is described above, and the radio wave transmitting and receiving method in the sensor system 1 according to one embodiment illustrated in the flowchart of FIG. 2, the sampling frequency setting part 4a and the vibration frequency upper limit identifying step S102 recognize the frequency range of the vibration to be superposed on the reflected waves of radio waves as noise, which are received by the radio wave sensor 2, and identify the upper limit of the frequency of the vibration to be superposed as noise. Subsequently, the sampling frequency setting part 4a and the sampling frequency determining step S103 determine that the sampling frequency fₛ of radio waves that the radio wave sensor 2 emits is the sampling frequency fₛ whose Nyquist frequency fₙ is equal to the identified upper limit frequency fᵤₚₚₑᵣ. The sampling frequency setting part 4a and the sampling frequency setting step S104 set the sampling frequency fₛ of radio waves that the radio wave sensor 2 emits to the determined sampling frequency fₛ.

Accordingly, this enables the radio wave sensor 2 to emit radio waves with the sampling frequency fₛ that is determined based on the vibration that is actually detected by the vibration sensor 3. Because of this, it becomes possible to control the frequency range of radio waves emitted from the radio wave sensor 2. Therefore, it becomes possible to reduce the time of radiating radio waves from the radio wave sensor 2 and the time of operating an analog-to-digital conversion (ADC) circuit and the like of the signal processing device 4. As a result, radio waves with an excessively high sampling frequency fₛ like the one used in the prior art technology are not emitted from the radio wave sensor 2. Thus, electric power is not consumed wastefully, and electric power consumption of the sensor system 1 can be reduced.

FIG. 4 is a perspective view illustrating an interior of a vehicle 11 that includes the sensor system 1 described above as a driver monitoring system (DMS).

In the case where the sensor system 1 is included in the vehicle 11 described above, the radio wave sensor 2 is installed in a back part 11a of a seat, a seat part 11b of the seat, a dashboard 11c, a ceiling 11d of the vehicle interior, or the like, and using a human body sitting in the seat as the measuring object 5, radio waves are radiated (emitted) to the human body. Further, the vibration sensor 3 is installed in the back part 11a of the seat, the seat part 11b of the seat, a floor 11e of the vehicle interior, or the like.

In the case where the vibration sensor 3 is installed in the back part 11a or the seat part 11b of the seat, a body motion of the human body or vibration of the vehicle is measured as the vibration, and in the case where the vibration sensor 3 is installed in the floor 11e of the vehicle interior, the vibration of the vehicle is measured.

According to the present configuration, it becomes possible to provide the vehicle 11 including the sensor system 1 that can reduce electric power consumption.

Note that the sensor system 1 can also be configured of a radio wave sensor, a vibration sensor, and an CPU included in a wearable device such as a smart watch or a smartphone. In this case, the radio wave sensor, the vibration sensor, and the CPU included in a wearable device or a smartphone function as the radio wave sensor 2, the vibration sensor 3, and the signal processing device 4 illustrated in FIG. 1, respectively. Further, the program of the flowchart illustrated in FIG. 2 is, for example, downloaded from the Internet network or the like as an application and installed in the wearable device or the smartphone.

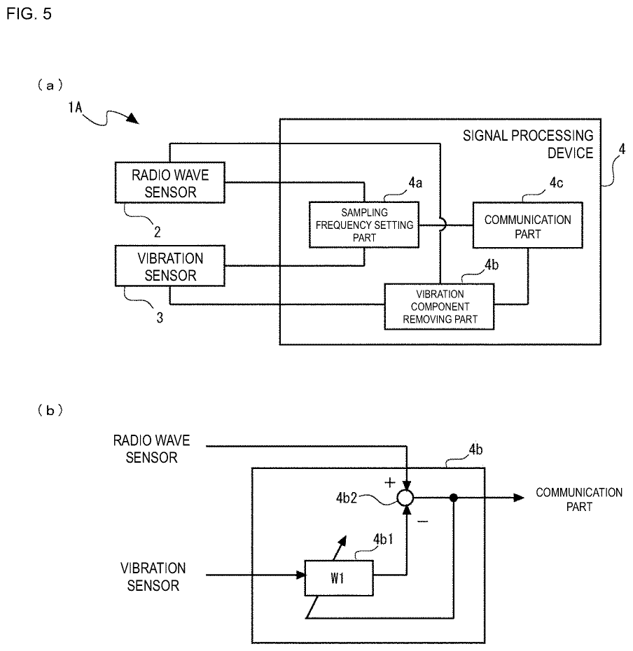

FIG. 5(a) is a block diagram illustrating a schematic configuration of a sensor system 1A according to a second embodiment of the present disclosure. The sensor system 1A is different from the sensor system 1 according to the first embodiment in that the signal processing device 4 is configured so as to include a vibration component removing part 4b and a communication part 4c, and the remaining part of the sensor system 1A is substantially the same as that of the sensor system 1 according to the first embodiment.

The vibration component removing part 4b attenuates or removes the component of the vibration data, which is noise measured by the vibration sensor 3, from the reflected wave data received by the radio wave sensor 2. In the case where the measuring object 5 is for example the human body and the reflected wave data includes the body surface displacement of the human body, this attenuation or removal enables the extraction of the body surface displacement of the human body. The communication part 4c transmits extracted body surface displacement data to an external device such as a personal computer, an ECU (Electronic Control Unit) of the vehicle, or the like using wired or wireless connection. In the external device, a predetermined process that uses received body surface displacement data or the like is performed.

The vibration component removing part 4b separates the vibration data input from the vibration sensor 3 from the reflected wave data input from the radio wave sensor 2 using a blind audio source separation technique such as Independent Component Analysis (ICA) or Independent Vector Analysis (IVA). In this case, the dimension of the reflected wave data is m in the International System of Units, and the dimension of the vibration data is m/s² in the International System of Units. Thus, an arithmetic process in the vibration component removing part 4b is to perform the independent component analysis or the independent vector analysis after integrating the vibration data twice.

Further, the vibration component removing part 4b may be an adaptive filter that uses an algorithm such as LMS (Least Mean Square) or the like. The adaptive filter can separate the vibration data input from the vibration sensor 3 from the reflected wave data input from the radio wave sensor 2. FIG. 5(b) is a block diagram illustrating a circuit configuration of the vibration component removing part 4b that is configured using an adaptive filter 4b1 of this type.

The noise (vibration data) having been superposed on the reflected wave data input from the radio wave sensor 2 is superposed after propagating a separated distance from the source of the noise, and thus the noise is affected by transfer characteristics therebetween. Therefore, the vibration component removing part 4b obtains a difference between the reflected wave data and the vibration data using a subtractor 4b2 and feed backs this difference to the adaptive filter 4b1 from an output of the subtractor 4b2. Subsequently, the vibration component removing part 4b adjusts the magnitude of a transfer coefficient W1 of the adaptive filter 4b1, separates the vibration data from the reflected wave data, and extracts the body surface displacement or the like.

Further, the vibration component removing part 4b may be Demucs, Sepformer, Conv-TasNet, or the like, which is used for audio separation, or may be the one that uses a method of a machine learning technique based on a modified version of Demucs, Sepformer, or Conv-TasNet. These methods enable the separation of the vibration data input from the vibration sensor 3 from the reflected wave data input from the radio wave sensor 2.

With the sensor system 1A according to the second embodiment described above, it becomes possible to attenuate or remove the component of the vibration data measured by the vibration sensor 3 from the reflected wave data received by the radio wave sensor 2 with reduced electric power consumption and without changing the ability of removing the vibration component. Further, as described above using FIG. 4, the sensor system 1A according to the second embodiment is also applicable to the driver monitoring system by appropriately arranging the radio wave sensor 2 and the vibration sensor 3 in the vehicle interior of the vehicle 11. In this case, it becomes possible to provide the vehicle 11 including the sensor system 1A that can reduce electric power consumption without changing the ability of removing the vibration component.

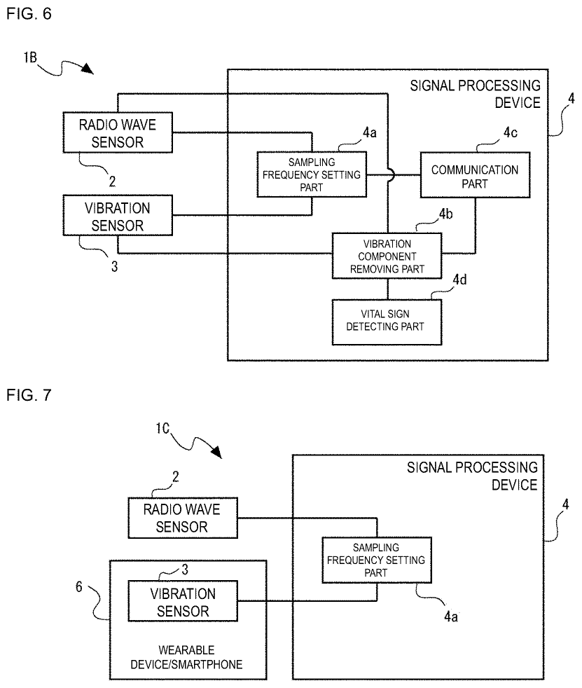

FIG. 6 is a block diagram illustrating a schematic configuration of a sensor system 1B according to a third embodiment of the present disclosure. The sensor system 1B is different from the sensor system 1A according to the second embodiment in that the signal processing device 4 is configured so as to include a vital sign detecting part 4d and the measuring object 5 is a human body, and the remaining part of the sensor system 1B is substantially the same as that of the sensor system 1A according to the second embodiment.

The vital sign detecting part 4d detects a vital sign of the human body from the reflected wave data in which the component of the vibration data is attenuated or removed by the vibration component removing part 4b. The vital signs include a heart rate, a heart rate variability, a respiration rate, a depth of respiration, and the like of the human body, which is the measuring object 5. Based on the reflected wave data input from the radio wave sensor 2, the vital sign is detected from the body surface displacement of the human body that is measured as a function of the distance from the radio wave sensor 2 to a radio wave irradiation part on the human body.

According to the sensor system 1B according to the third embodiment described above, without changing the ability of removing the vibration component from the vital sign, it becomes possible to detect a vital sign of the human body with reduced electric power consumption from the reflected wave data whose vibration component is attenuated or removed by the vibration component removing part 4b. Further, as described above using FIG. 4, the sensor system 1B according to the third embodiment is also applicable to the driver monitoring system by appropriately arranging the radio wave sensor 2 and the vibration sensor 3 in the vehicle interior of the vehicle 11. In this case, it becomes possible to provide the vehicle 11 including the sensor system 1B that can reduce electric power consumption without changing the ability of removing the vibration component from the vital sign.

FIG. 7 is a block diagram illustrating a schematic configuration of a sensor system 1C according to a fourth embodiment of the present disclosure. The sensor system 1C is different from the sensor system 1 according to the first embodiment in that the vibration sensor 3 is configured using a built-in vibration sensor of a wearable device such as a smart watch or the like or a built-in vibration sensor of a smartphone 6. The remaining part of the sensor system 1C is substantially the same as that of the sensor system 1 according to the first embodiment. The wearable device or smartphone 6 is not limited thereto and may alternatively be any mobile device having the vibration sensor 3 and communication capability. In the present configuration, the communication between the vibration sensor 3 and the signal processing device 4 is performed using a wired or wireless connection such as Bluetooth (registered trademark) or the like.

The sensor system 1C according to the fourth embodiment enables the radio wave sensor 2 to emit radio waves with the sampling frequency fₛ obtained based on the vibration actually detected using the built-in vibration sensor of the wearable device or smartphone 6. Therefore, it becomes possible to provide the sensor system 1C that can simplify the configuration of the sensor system 1C, reduce prices of products each including the sensor system 1C, control the frequency range of radio waves emitted from the radio wave sensor 2, and reduce electric power consumption.

Note that as is the case with the sensor system 1C according to the fourth embodiment, in the sensor system 1A according to the second embodiment and the sensor system 1B according to the third embodiment, the vibration sensor 3 can be configured using the built-in vibration sensor of the wearable device such as a smart watch or the like or the smartphone 6. Even in such cases, substantially the same actions and effects as those of the sensor system 1C according to the fourth embodiment are produced. Further, as described using FIG. 4, the sensor system 1C according to the fourth embodiment is also applicable to the driver monitoring system by appropriately arranging the radio wave sensor 2 and the wearable device or smartphone 6 that serves as the vibration sensor 3 in the vehicle interior of the vehicle 11. Even in this case, it becomes possible to provide the vehicle 11 including the sensor system 1C that can reduce electric power consumption.

### REFERENCE SIGNS LIST

1, 1A, 1B, 1C Sensor system
2 Radio wave sensor
3 Vibration sensor
4 Signal processing device
4a Sampling frequency setting part
4b Vibration component removing part
4c Communication part
4d Vital sign detecting part
5 Measuring object
6 Wearable device or smartphone

1. A sensor system comprising:
a radio wave sensor that emits an electromagnetic wave toward a measuring object and receives a reflected wave reflected assuming the electromagnetic wave hits the measuring object;
a vibration sensor that measures vibration to be superposed on the reflected wave as noise; and
a sampling frequency setting part that recognizes a frequency range of the vibration, identifies an upper limit of the frequency of the vibration, determines that a sampling frequency of the electromagnetic wave that the radio wave sensor emits is a sampling frequency whose Nyquist frequency is equal to an identified upper limit of the frequency, and sets the sampling frequency of the electromagnetic wave that the radio wave sensor emits to a determined sampling frequency.

2. A radio wave transmitting and receiving method comprising:
a vibration measuring step that emits an electromagnetic wave toward a measuring object from a radio wave sensor and measures vibration to be superposed on the reflected wave reflected assuming the electromagnetic wave hits the measuring object as noise;
a vibration frequency upper limit identifying step that recognizes a frequency range of the vibration and identifies an upper limit of the frequency of the vibration;
a sampling frequency determining step that determines that a sampling frequency of the electromagnetic wave that the radio wave sensor emits is a sampling frequency whose Nyquist frequency is equal to an identified upper limit of the frequency; and
a sampling frequency setting step that sets the sampling frequency of the electromagnetic wave to the sampling frequency determined by the sampling frequency determining step.

3. The sensor system according to claim 1, further comprising:
a vibration component removing part that attenuates or removes component of the vibration measured by the vibration sensor from the reflected wave.

4. The sensor system according to claim 3, further comprising:
a vital sign detecting part, wherein
the measuring object is a human body, and
the vital sign detecting part detects a vital sign of the human body from a reflected wave in which the component of the vibration is attenuated or removed by the vibration component removing part.

5. The sensor system according to claim 4, wherein
the vibration sensor is a built-in vibration sensor of a wearable device.

6. The sensor system according to claim 4, wherein
the vibration sensor is a built-in vibration sensor of a smartphone.

7. A vehicle comprising the sensor system according to claim 6.

8. The sensor system according to claim 1, wherein
the vibration sensor is a built-in vibration sensor of a wearable device.

9. The sensor system according to claim 1, wherein
the vibration sensor is a built-in vibration sensor of a smartphone.

10. The sensor system according to claim 3, wherein
the vibration sensor is a built-in vibration sensor of a wearable device.

11. The sensor system according to claim 3, wherein
the vibration sensor is a built-in vibration sensor of a smartphone.

12. A vehicle comprising the sensor system according to claim 1.

13. A vehicle comprising the sensor system according to claim 3.

14. A vehicle comprising the sensor system according to claim 4.

15. A vehicle comprising the sensor system according to claim 5.
